(19) United States
(12) Patent Application Publication
(10) Pub. No.: US 2021/0113072 A1
(43) Pub. Date: Apr. 22, 2021

(54) BALA 2/3 DIMENSION ENDOSCOPE

(71) Applicant: John L. Bala, Hollywood, SC (US)

(72) Inventor: John L. Bala, Hollywood, SC (US)

(21) Appl. No.: 16/542,474

(22) Filed: Oct. 21, 2019

(51) Int. Cl. A61B 1/00; A61B 1/05; A61B 1/07; A61B 1/06

(52) U.S. Cl. CPC A61B 1/00193; A61B 1/00096; A61B 1/051; A61B 1/00048; A61B 1/00016; A61B 1/0684; A61B 1/07

(57) ABSTRACT

The Bala 2/3 Dimensional Endoscope generates either two or three dimensional images from a rigid or flexible endoscope that is approximately 4 millimeters in diameter. The endoscope provides a means selecting from a switch on the device to select either a two or three dimensional view from within the body cavity without removing the endoscope from the body and requires no external light source for illumination.

### BACKGROUND OF THE INVENTION

Endoscopes are devices used in the field of medicine that allow visual examination of muscles, bones, organs and tissue for diagnostics, sampling of tissue, safe manipulation of instruments and for performing minimally invasive surgery. The term “Endoscope,” can be applied to a wide range of visualization devices such as arthroscope, cystoscope, laryngoscope, etc. that are used for device related procedures.

In most cases the instruments used to examine the body are optical devices that create a two dimensional image that is viewed by the surgeon on a video monitor. These endoscopes are optical instruments consisting of a telescope for viewing the image and a light source to provide visible light for illumination of the subject area of interest. In many cases, an electronic camera is connected to the telescope to display images from within the body cavity on a video monitor. Three dimensional endoscopes are available using very complex dual optical path telescopes that optically combine the two paths in order to view in three dimensions.

The Bala 2/3 Dimensional Endoscope simulates visual activity that occurs between the eyes and the brain when it processes images. In effect, the brain converts what two eyes see independently into three dimension stereo pictures in the brain. The Bala 3 Dimensional Endoscope simulates the 3D effect using two individual micro cameras, each spaced at a slightly different distances from each other with each camera recording the independently recording the same image of the subject simultaneously and individually. The cameras send the two separate images to a computer, much like the human eye sends images to the brain. The individual images are created at frame rates that project a continuous image using a frame rate of approximately 30 images/second/camera, thereby, generating two real time dynamic images that are blended by computer processing into one 3D image.

An objective lens integrated with each camera integrated circuit, located at the distal end of the device, record simultaneous images by means of a strobe effect which acquires and freezes images from each camera during illumination of the subject area. The strobe is created by pulsed light emitting diodes (LEDs) which illuminate the subject area viewed by the two cameras. Synchronized controls activate the cameras to record each image during the time that the image area is illuminated. The two recorded images are stored and processed by the computer into a singular three dimensional image to be viewed on a video monitor as a pseudo 3D display, or can viewed in depth with a head mounted holographic projection system.

The camera/objective cameras are spaced to give the same stereo 3D image similar to the images using stereo viewer. The Bala 3 Dimensional Endoscope uses two images from two cameras viewing the same object at critical spacing differences to generate 3D stereo effect after post computer processing.

The camera integrated circuit outputs are connected to a multiple conductor cable which transmits control functions and data to and from the cameras to circuitry located at the proximal end of the device where it is processed into a singular 3D digital image.

Integral to the recording by the cameras is the illumination provided from the proximal end of the device by LEDs embedded into a conical plastic condenser element and connected to an array of fiber optics that transmit the light to a distal assembly consisting of the two cameras surrounded by the illumination fiber optics randomly cemented together surrounding the cameras. The data cable transits the center of the conical lens and is surrounded by the individual optic fibers and contained within a stainless steel tube or flexible encapsulation.

The LED assembly consist of a circular array of individual LEDs embedded into the conical lens at one end and a annulus ring of fibers optics at the other. The center of the conical lens is cored out to allow the electric cable to transit from the distal to the proximal end of the endoscope. The conical lens acts like a large fiber optic with a high index of refraction core material and a low index cladding. It is tapered to condense the light from a array of LEDs match the entrance angle of the individual fiber elements. Four LEDs embedded into the conical condenser provide the light to illuminate the subject area with enough energy to encompass the dynamic range of the camera's sensitivity.

Another embodiment of the illumination of the Bala 2/3 Dimensional Endoscope utilized a high index optical grade plastic in place of the glass lens used as the condenser. The high index plastic lumen conducts the light from the LEDs conical condenser to the individual fibers cemented to the annulus proximal end and assembly. The high index plastic material is the core and air is the low index cladding normally associated with fiber optics. Although the Numerical Aperture is less than that of glass fiber, it none the less provides sufficient light to utilize the full dynamic range of the electronic cameras.

The proximal end encompasses the processing electronics image data storage to converge the two independent images in to one singular 3D image. A miniature USB 3 connector and cable transmits the output image the desire video display device. The connector also provides the power for the device.

In many cases the surgeon may prefer a two dimensional visualization to work his way through body passages, particularly with a flexible endoscope to locate a polyp or tumor. He may then wish to generate a more comprehensive three dimensional image to perform an actual procedure or choose to switch back and forth to gain a perspective. The surgeon may find the use the two dimension image to guide instruments from an entry incision to the surgical site or may even switch imaging conditions should one of the cameras become obscured by blood or tissue or disabled. The ability to switch can be accomplished in a relatively short time without removing the device from the surgical site.

The three dimensional endoscope can easily be created to a two dimensional endoscope by shutting off one of the cameras and process the image in two dimensions. The embodiment of the Bala 2/3 Dimensional Endoscope provides for this capability by providing a means of switching the hardware and software to provide this capability.

### SUMMARY OF THE BALA 3 DIMENSIONAL ENDOSCOPE INVENTION

The embodiment of the Bala 2/3 Dimensional Endoscope consists of two miniature cameras with integral objective lenses mounted side by side separated to give two separate images. Using digital processing results in a three dimensional image created as a pseudo 3D video display or projected on a 3D holographic head mounted display. The two cameras are surrounded by an array of individual fiber optics used to illuminate the subject area with visible light from LEDs located at the proximal end of the endoscope.

In the preferred embodiment, two 1.8×1.8 video camera chips mounted on a single ceramic substrate are enclosed in a thin walled enclosure that isolate the cameras from the individual illuminating fiber optics that surround the assembly to prevent cross talk between the illumination and the imaging elements. In the embodiment the integrated camera circuits are bonded to a ceramic substrate for mechanical retention, electrical interconnection and positioning. A second substrate is used to bond electrical wired connections to a multi conductor cable to connect the cameras to the electronic controls that flow to and from a computer processing element located at the proximal end. In the preferred embodiment the two substrates are bonded to the same substrate to create a complete camera assembly.

The multi conductor cable passes the length of the endoscope, in which the length of the endoscope may vary based on the application and use. The cable passes through the core of an optical conical condenser lens element and connects to a computer processing unit that stores the individually recorded images as data in an intermediate step to creating the 3D image.

In the preferred embodiment a conical tapered condenser lens element made of a high transmission, high index optical grade plastic captures the output of an array of LED's that are embedded into the surface of the conical condenser and are cemented into the plastic condenser lens with an index matching cement. Another embodiment using a glass conical lens made as a tapered fiber optic with high and low index materials could be used, but would create a more costly assembly.

In the preferred embodiment, the plastic tapered conical lens would be molded from the high index, high transmission optical grade plastic and the surfaces highly polished. Using the high index as the core material for the conical lens and air as the low index medium of the conical condenser lens would create a fiber optic effect of total internal reflection.

In the preferred embodiment the light from the LEDs would be collected by the condenser lens and exit at the narrow end of the taper conical lens creating an annulus in which randomized individual fibers optics would be cemented. The internal surface of the hollowed tapered condenser lens would be at the same angle as that of the outer diameter of the lens. The hollowed core creates a means for the electronic cable to pass through the lens to its termination. Individual fibers surround the electronic cable as they transit the length of the device from the condenser lens and are bundled together surrounding the camera assembly. In the embodiment, the distal end fiber optics are cemented together, polished and retained.

The entire assembly of the cameras and illumination fiber optics are protected by an external sheath, either of stainless steel for rigid applications or a flexible sheath for endoscopes requiring articulation. At the proximal end of the device, a miniature electrical connector is provided for power and data transmission. However, the embodiment does not preclude the use of wireless technology to transmit the 3D image for viewing and recording purposes. A electrical switch at the proximal end of the endoscope is used to switch back and forth from 2D to 3D.

In the preferred embodiment, the system is electronically controlled by a series of commands to and from the electronic controls of the endoscope. A command to energize the endoscope powers an array of LED illumination lights surrounding the conical condenser lens to turn on. This command that powers the LEDs for the time necessary to record the images in the electronic cameras. The same command initiates the simultaneous recording of images from both cameras. The cycle time for this operation is approximately 30 times a second in order to provide the appearance of a continuous image. The pulsing of the LEDs acts as a strobe light and assures that both cameras are synchronized to visualize each image for the same exact period of time. The LEDs operating in the pulsed mode generate more light output per pulse over operating the LEDs in a continuous mode.

Once the images are captured, the LEDs are turned off until the next cycle and no further recording of images occurs. During the off cycle, the images are transmitted and processed by a computer and the output is transmitted to a viewing or recording device. Transmitting, computing and displaying images all occur in overlapping sequences. For recording two dimensional images only one camera is used during the cycle period.

In the preferred embodiment, the 3D image output is displayed on a 3 Dimensional head mounded display which projects a holographic 3D image. Another embodiment is to display the 3D image on a pseudo 3D image on a standard video monitor. Furthermore, images may be recorded on other video or digital medium for viewing at a later time.

In another embodiment, the endoscope can be used to record two dimensional images, by selecting one of the two cameras. The selection of 2 or 3D is made by a switch located in the body of the endoscope at the proximal end. This capability can provide the physician with different perspectives of the image and in some cases be more functional and familiar than the 3 D image.

The endoscope provides new and expanded capabilities to the surgeon with a small diameter 2/3D device that can be used in a wide range of medical applications and at a cost that does not require a physically complex optical telescope with additional optics necessary to create a 3D image. In addition, it does not require a separate external illumination system to provide viewing light to the subject area being viewed. The use of micro cameras in place of coherent fiber optics generates a higher resolution image than device using coherent fiber arrays for imaging.

### BRIEF DESCRIPTION OF THE DRAWINGS

FIG. 1 is an orthogonal view of the embodiment of the Bala 2/3 Dimensional Endoscope.

FIG. 2 is an expanded view of the distal camera and illumination systems.

FIG. 3 is an expanded view of the Conical Condenser lens, LED Illumination and Electronic layout.

FIG. 4 is a flow diagram of the 3 Dimensional Endoscope.

FIG. 5 is a flow diagram of the 2 Dimensional Endoscope.

### DETAILED DESCRIPTION OF THE INVENTION

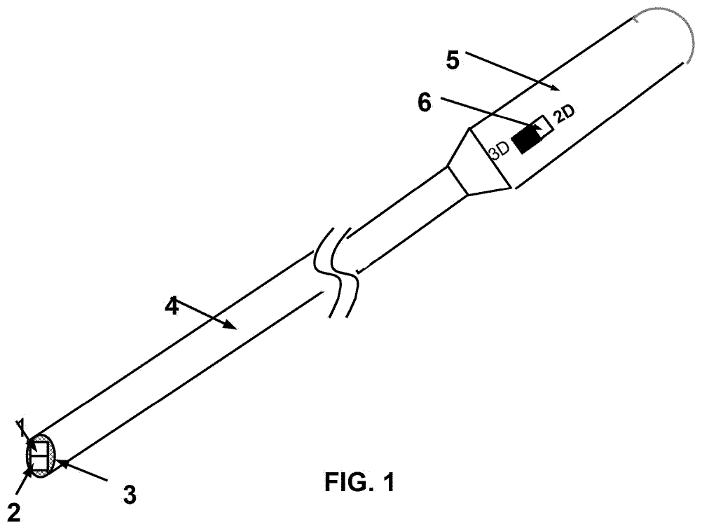

A preferred embodiment of the invention illustrated in FIG. 1 is the Bala 2/3 Dimensional Endoscope.

The illustration is of two electronic cameras, 1 & 2 with integrated objective lenses.

The two electronic cameras 1 & 2 are spaced from each other to record two images of the same subject by a pre defined separation.

An array of fiber optics used for illumination 3, surround the electronic camera. The assembly of 1,2,& 3 make up the distal sensing and illumination elements for creating 2 & 3D imaging.

In the preferred rigid embodiment, the distal assembly of 1,2 & 3 are housed in a stainless steel tube, 4 that contain the individual fiber optic illumination 3 and the electronic cables that transmit signal to and from the electronic camera 1 & 2 and the light to the fiber optic illumination 3. In the preferred embodiment the length of the stainless steel tube 4 s defined for a given medical application.

In another embodiment, the distal assembly 1,2 & 3 is encompassed in a flexible retainer, the length again defined for a given medical application.

The proximal assembly 5, contains the optical and electronic elements necessary to process the individual images from the cameras 1 & 2 and to convey those images to a means to create a video view of a 2 or 3D image.

In the embodiment, a switch, 6 located at the proximal end of the Bala 2/3 Dimensional Endoscope provides a means for the user to switch the output image to either 2 or 3 D.

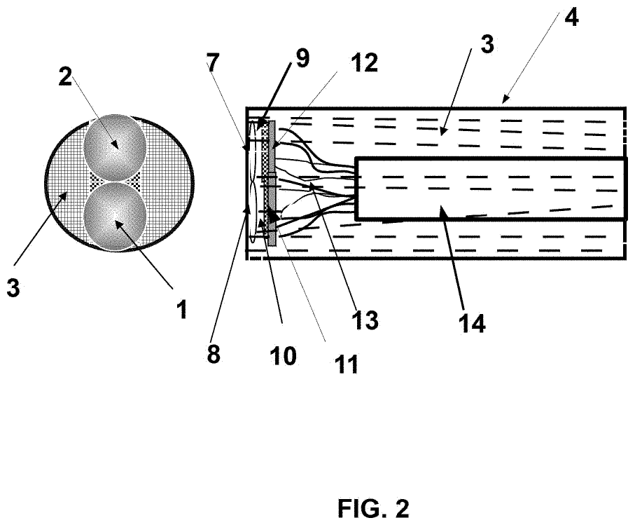

FIG. 2 is a frontal and side view of the distal end of the Bala 2-3 Dimensional Endoscope.

In the preferred embodiment, the electronic cameras 1 & 2 are shown in greater detail. The cameras are surrounded by individual fiber optic illumination elements 3 are bonded together, ground and polished and cemented together with the camera elements 1 & 2.

In the preferred embodiment, the entire assembly is cemented to a stainless steel tube, 4 whose length is determined by the medical application to which it will be applied.

In the embodiment, the objective lenses 7, & 8 are bonded to the camera integrated circuit chip 9 & 10 and then to a ceramic substrate 11. This substrate is again bonded to another substrate that is connected to an electrical cable, 12 that transmits data to and from the camera integrated circuits via the electronic connections 13 and cable 14 to controls located in the proximal assembly 5.

In the preferred embodiment the fiber optics that surround the camera, 3 transit the length of the distal end to interface and cemented to a conical condenser lens mounted in the proximal assembly FIG. 1, 5.

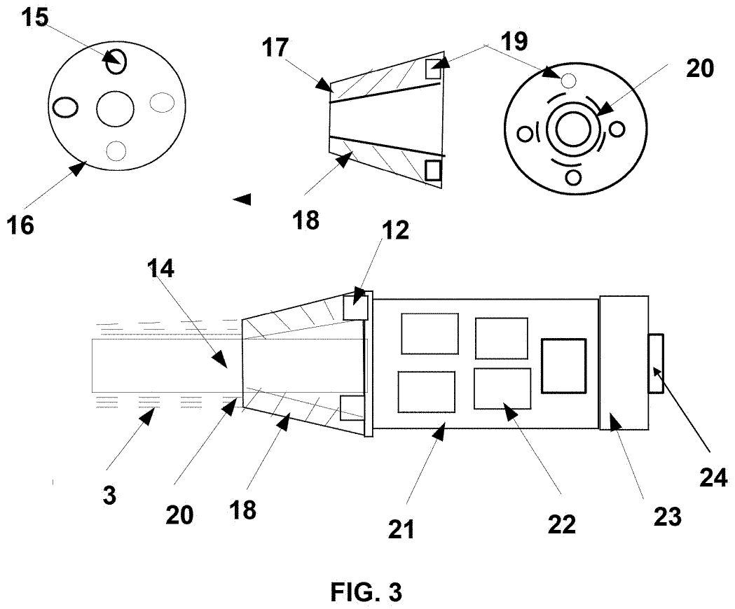

FIG. 3 is a sectional view of the proximal part of the Bala 2/3 Dimensional Endoscope.

In the preferred embodiment the optical illumination fibers, 3 are cemented to the annulus 17 of the conical condenser lens 18. These fibers transit the length of the device and terminate at the distal front surface FIG. 1, 3.

In the embodiment, the conical condenser lens, 18 is a form of fiber optic. The Fiber optic is made to act like a condenser lens by means of total internal reflection of light from four LEDs, 15 that are integrated in to the conical lens assembly 18 at indentations made in the lens, 25 and cemented into the conical lens 18 with an index matching cement. The electrical connections to the LEDs are made through a circuit board 16.

The center of the conical condenser lens 18, is cored out to allow the electrical cable, 14 to pass through the center of the lens and connect to the electrical assembly 21.

In the embodiment, the electronic circuits, 22 process the image data transmitted by the cameras, FIG. 2, 1 & 2 through the electrical cable and are processed by the electronic circuitry. In addition commands to the cameras are processed by the electronic circuitry in a hand shaking mode of data going to the cameras 1 & 2 and data coming from the cameras 1 & 2 to the circuitry.

In the embodiment, the electronic circuitry activates the LEDs, 15 at the same time that the cameras 1 & 2 are processing image information from the subject area; thus, synchronizing the camera's 1 & 2 with the LEDs, 15.

In the embodiment, cameras 1 & 2 see essentially the exact same image at the same time, separated in distance in order that computer software process the two images creating a stereo digital three dimensional image that can be viewed as a pseudo 3D video image or by a head mounted holographic 3D viewer.

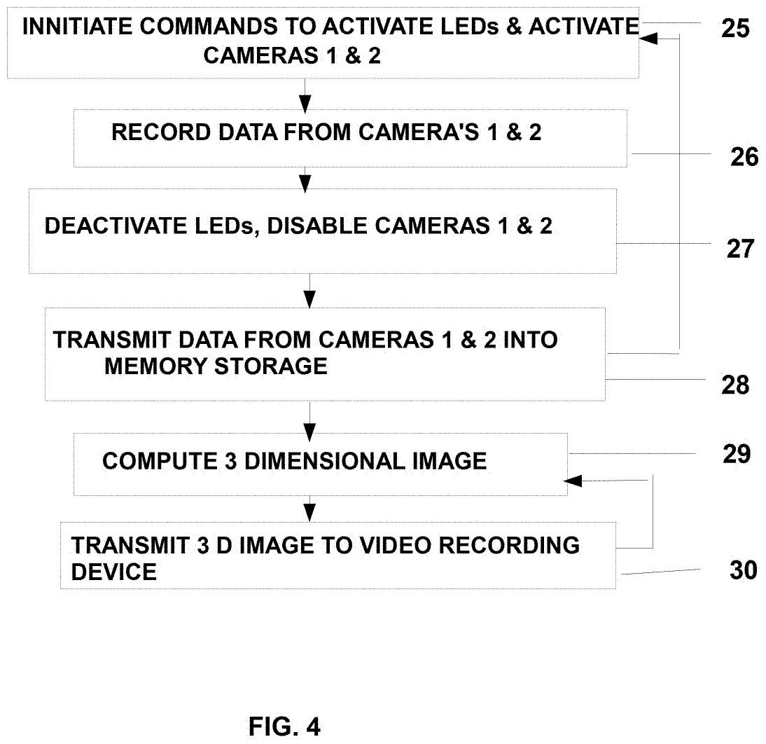

FIG. 4 is a flow chart of the embodiment of the 3 dimensional endoscope.

The three dimensional endoscope is initiated by electronic commands to activate the four LED's, 25 and to activate cameras 25. This process captures two images spaced within a specific distance simultaneously and transmits the digital images recorded by the cameras to electronic storage associated with the processing of each image. The process is a synchronized system whereby cameras FIGS. 1 & 2 capture two images that appear to be the same image.

Once the data has been recorded the image data from each of the cameras is stored into individual digital processors, 26 at which time the LEDs and cameras are de activated 27.

The recording cycle, 27 is complete when the digital data from the cameras has been transmitted into electronic storage of the computer that combines the individual camera images into a singular three dimensional image, 28. At the complete of the transmission, the computer releases the system to initiate a new recording cycle 25 and proceed to compute a three dimensional image, 29.

The electronic processing is given to re initiate a repeat cycle, 25 at approximate 30 frames per second to provide the effect of real time images. Once a three dimensional image is computed, 29 it transmits the three dimensional image to a video recording device such as a heads up holographic display, pseudo 3D video display or as data to be stored for archival records or data to be printed on hard copy. Once one image is recorded, the processor returns to generate the next image, 30.

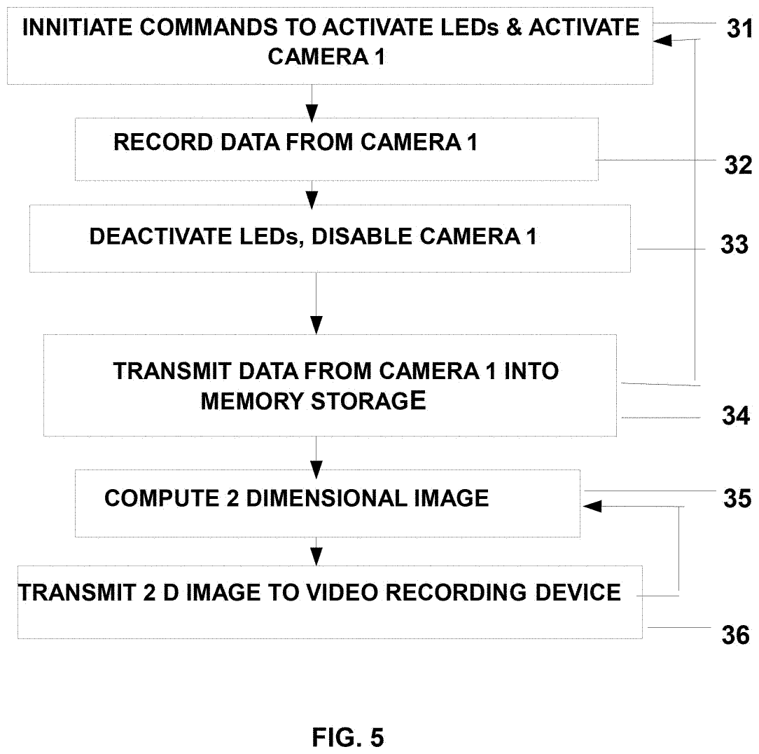

FIG. 5 is an embodiment of the same three dimensional imaging system using only one camera to produce a two dimensional view of the subject image. When a switch, FIG. 1, 6 is activated on the proximal housing, a selection of a two dimensional view is made. With the switch in the two dimensional position, the system performs according to the cycle outlined in FIG. 4, and executes the image retrieval executing the function 31 to 36 utilizing one camera to provide a two dimensional display of the image on a video monitor, consistent with the images obtained with current two dimensional endoscopes.

1-14. (canceled)

15. A 2/3 Dimensional Endoscope comprising:
a metal or plastic tube, either rigid or flexible having a distal (front) and proximal (rear) end;
two spatially separated image sensors with an objective lens integrated with each sensor within said tube;
an electronic data processing system to collect and process image data from said individual image sensors to generate two or three dimensional digital equivalents of said images;
wherein, individual fiber optic elements comprised of individual strands of optical fibers which are bonded together in the form a ring that is bonded onto the small diameter of a fiber optic conical condenser lens at the proximal end of said tube and terminating as a bundle of individual fibers bonded together at the distal end of said tube;
an electronic cable is surrounded by said strands of individual optical fibers and contained within the said tube;
a fiber optic conical lens located at the proximal end having a hollow core center to enable said electronic cable to pass through the center of said fiber optic conical lens;
an array of light emitting diodes (LEDs) embedded into the top surface of the larger diameter of said conical condenser lens surface;

16. The endoscope of claim 15, wherein a fiber optic conical lens constructed with the inner surface of the core and outer surface of the lens are parallel.

17. The endoscope of claim 15, wherein a fiber optic conical lens condenses light present at the larger diameter lens surface to the smaller diameter lens surface by total internal reflection.

18. The endoscope of claim 15, wherein, the proximal ends of individual strands of optical fibers optics are bonded together in a ring and bonded to the smaller diameter of said fiber optic conical lens.

19. The endoscope of claim 15, wherein said two identical imaging sensors have overlapping fields of view and store two separate images by synchronizing the activation of said LEDs and image storing by said electronic data processing system at exactly the same time and distance of two spatially separated images.

20. The endoscope of claim 15, wherein electronic data processing synchronizes each of the said sensors with an electronic command to activate said LED illumination for a given period of time to record data from said sensors and wherein they are electronically combined by electronic data processing into a single 3 dimensional digital image.

21. The endoscope of claim 15, wherein a multi function integrated switch with a selector on said tube commands said electronic data processing system to adjust the brightness level of said LEDs.

22. The endoscope of claim 15, wherein said a multi function integrated switch mounted on said tube selects one of two image sensors to command said electronic data processing system to process a 2 dimensional image.

23. The endoscope of claim 15, wherein said fiber optic conical lens located at the distal end of said tube includes LEDs which are embedded into the larger diameter of the lens are cemented into said lens with an index matching cement to avoid surface reflection losses.

25. The endoscope of claim 15, wherein a two or three dimensional digital image are stored within the electronic processing system from said image sensors and formats the data consistent with a heads up display and a pseudo three dimensional image on a video monitor, or as a two dimensional image on a video monitor.
